(19) United States
(12) Patent Application Publication
(10) Pub. No.: US 2019/0247851 A1
(43) Pub. Date: Aug. 15, 2019

(54) CAPILLARY-LOADED ANALYSIS DEVICE FOR BIOLOGICAL FLUID SAMPLES

(71) Applicant: Athelas, Inc., Saratoga, CA (US)

(72) Inventor: Louis M.N. Virey, San Francisco, CA (US)

(21) Appl. No.: 16/274,136

(22) Filed: Feb. 12, 2019

Related U.S. Application Data
(60) Provisional application No. 62/629,557, filed on Feb. 12, 2018.

(51) Int. Cl. B01L 3/00; B01L 9/00

(52) U.S. Cl. CPC B01L 3/502715; B01L 9/527; A61B 5/150755; B01L 2400/0406; B01L 3/502769

(57) ABSTRACT

Analysis devices have structural features for capturing biological fluid samples and presenting them in a way allowing imaging of components. The analysis devices may include a capillary channel coupled to an inlet for receiving the biological sample. The capillary channel allows the sample to be drawn in by capillary force and form the suspension. It may have a precise and uniform height of, e.g., about 5 and 30 micrometers. For various cell types, height allows a single layer of unstacked cells to be presented for easy imaging at a single depth of focus. In certain embodiments, the analysis devices include a reservoir for initially holding the biological sample taken from a patient such as by drawing blood from a wound.

### CROSS-REFERENCE TO RELATED APPLICATIONS

This application claims benefit of U.S. Provisional Patent Application No. 62/629,557, filed Feb. 12, 2018, which is incorporated herein by reference in its entirety and for all purposes.

### BACKGROUND

Cell counting and characterization in biological samples is a commonly used procedure for clinical studies and for disease diagnostics, monitoring, and maintenance. For more frequent monitoring, cell counting is increasingly conducted outside of laboratory and clinical settings. Yet, some traditional methods for analyzing samples employ a microscopy setup operated by a trained operator. And some methods employ automated instruments that are very expensive and bulky. Furthermore, lay people including patients are sometimes asked to collect the biological fluid samples, hence the need for simple and inexpensive analysis devices that require little or no sample preparation.

### SUMMARY

This disclosure pertains to devices, sometimes referred to as analysis devices, for collecting blood or other biological fluids and producing a thin suspension that allows imaging of components such as cells, bacteria, or other microscopic particles in the samples, sometimes with little or no preparation. The analysis device may include a capillary channel coupled to an inlet for receiving the biological sample. The capillary channel allows the sample to be drawn in by capillary force and form the thin suspension. In certain embodiments, the analysis devices include a reservoir for initially holding the biological sample taken from a patient such as by drawing blood from a wound. In certain embodiments, the reservoir is shaped as a notch that passes fully or partially through the perimeter of the analysis device. In operation, a patient may place their pricked finger against the reservoir to allow rapid blood capture.

One aspect of this disclosure pertains to an analysis device for producing a thin layer of a liquid biological sample. The analysis device may be characterized by the following features: (a) a capillary channel having (i) an inlet for receiving the biological sample, (ii) a height sized to form a thin layer of the biological sample when the biological sample enters the capillary channel, and (iii) a width (which may be a diameter in embodiments employing a round channel), in a direction substantially perpendicular to flow of the biological sample at the inlet; (b) a substantially flat and transparent window defining a surface of the capillary channel and configured to form a thin layer suitable for imaging the biological sample through the substantially transparent window; and (c) a dye or other reagent coated on at least a portion of the substantially transparent window and/or another surface of the capillary channel, wherein the dye stains a particular cell type from the biological sample when the biological sample contacts the dye. The height of the capillary channel may be between about 5 and 30 micrometers. In certain embodiments, the width or diameter of the capillary channel may be between about 1 and 40 millimeters. While various characteristics of a capillary channel have been described here, some or all of these characteristics may be varied or adapted depending on the type of fluid to be collected. For example, some biological samples are more viscous than others. In alternative embodiments, the analysis device does not include a dye or reagent.

In certain embodiments, the height of the capillary channel is between about 10 and 15 micrometers. In certain embodiments, the height of the capillary channel is sized to form a monolayer of particles, for instance cells, in the biological sample. In certain embodiments, the capillary channel additionally includes a length, in a direction substantially parallel to a direction of flow of the biological sample at the inlet, of between about 5 and 40 millimeters.

In some implementations, the substantially transparent window includes more than one transparent layers. In some cases, the substantially transparent window has a thickness of between about 0.1 and 1.5 millimeters.

In designs employing top and bottom surfaces, both of them may be substantially flat. In some designs, the analysis device has a substantially flat surface, such that the substantially transparent window and the substantially flat surface are disposed parallel to one another and separated by the height of the capillary channel to thereby partially enclose the capillary channel. As an example, the analysis device may contain a separation structure that separates the substantially transparent window and the substantially flat surface by the height of the capillary channel. In certain embodiments, the separation structure includes double-sided adhesive tape.

The analysis device may additionally include a support frame for supporting the substantially transparent window and the capillary channel without interfering with imaging the monolayer. In other designs, the analysis device does not have a support frame. In such embodiments, the substantially transparent window may have one or more indentations sized and shaped to permit grasping with fingers.

In certain embodiments, the dye is or includes methylene blue and/or cresyl violet, which may be provided, for example, in the acetate form. In some cases, the analysis device additionally includes a lysing agent for one or more types of cells present in the biological sample. The lysing agent may be provided on at least a portion of the substantially transparent window and/or the other surface of the capillary channel. As an example, the lysing agent is a hemolysing agent such as Saponin. In some embodiments, the analysis device additionally includes a reagent that reduces adhesion of certain types of cells to at least a portion of the substantially transparent window and/or the other surface of the capillary channel.

In certain embodiments, the analysis device additionally includes a reservoir configured to receive the liquid biological sample, wherein the reservoir comprises a notch in (i) one or two parallel plates defining a portion capillary channel, and/or (ii) a frame of the analysis device, and wherein the reservoir is fluidically coupled to the inlet for receiving the biological sample.

Aspects of this disclosure pertain to methods of manufacturing analysis devices. In certain embodiments, the analysis device is fabricated by adhering two parallel plates to opposite sides of a double-sided adhesive, which serves as a separation structure defining the height of the capillary channel.

Another aspect of the disclosure pertains to a method of producing and/or using an imageable suspension such as a monolayer. The method involves applying a biological sample to an analysis device that includes the following elements: (a) a capillary channel having (i) an inlet for receiving the biological sample, (ii) a height sized to form a thin layer of the biological sample when the biological sample enters the capillary channel, wherein the height is between about 5 and 30 micrometers, and (iii) a width, in a direction substantially perpendicular to flow of the biological sample at the inlet, of between about 1 and 40 millimeters; (b) a substantially transparent window defining a surface of the capillary channel and configured to form a suspension suitable for imaging the biological sample through the substantially transparent window; and (c) a dye coated on at least a portion of the substantially transparent window and/or another surface of the capillary channel, wherein the dye stains a particular cell type from the biological sample when the biological sample contacts the dye. In some methods, the operation of applying a biological sample to an analysis device includes causing a patient to contact a wound with a reservoir on the analysis device. In some embodiments, the height of the capillary channel is defined by a layer of adhesive tape between two substantially parallel plates that define upper and lower surfaces of the capillary channel

In certain embodiments, the method additionally includes imaging the suspension to produce an image of the suspension. And, the method may further include applying the image of the suspension to an image processing algorithm to classify and count a given type of particles in the suspension. In one image or field of view of a suspension, where the image or field of view is parallel to the window, a particle concentration may be obtained as follows. For N particles observed on an image of dimensions h and w, the concentration C of particles in the sample can then be derived by the formula:

C=N/(d×h×w)

where d is the height of the capillary channel.

These and other features of the disclosed embodiments will be further described below with reference to the drawings.

### BRIEF DESCRIPTION OF THE DRAWINGS

FIGS. 1A and 1B provide upper side and underside perspective views of a fully assembled analysis device containing a frame in accordance with certain embodiments.

FIGS. 2 and 3 illustrate exploded and assembled views of transparent analysis device components including a frame in accordance with certain embodiments.

FIGS. 4 and 5 illustrate exploded and assembled views of transparent analysis device components without a frame in accordance with certain embodiments. The disclosed embodiments have a reservoir on a front side of the analysis device.

FIGS. 6A and 6B illustrate exploded and assembled views of transparent analysis device components without a frame in accordance with certain embodiments. The disclosed embodiments have a reservoir on a side of the analysis device.

FIGS. 7 and 8 illustrate generalized representations of certain analysis devices.

### DETAILED DESCRIPTION

### Introduction

Aspects of this disclosure pertain to devices, sometimes called sample analysis devices (or just analysis devices), disposable microfluidic cartridges, or consumables, for capturing and imaging a suspension of a test biological sample. When an analysis device is used to identify cells in a sample, it may be referred to as a counting-chamber device or a cell counter. The disclosed analysis devices allow one to image individual cells (including but not limited to blood cells) and other microscopic features inside a fluid (including but not limited to biological fluids). There are various applications of the analysis devices described herein. One such application prepares a thin suspension of whole blood including white blood cells and red blood cells. When used with an automated imaging analyzer (like those using image analysis procedures such as described in PCT Patent Application No. PCT/US17/14982, which is incorporated herein by reference in its entirety), it allows the patient to safely and easily do their own blood count. Of course, a person other than the patient (e.g., a clinician or other health care provider) can also take a sample and generate a count.

Traditionally, cell counts are performed in a lab using a haemocytometers (also used for sperm and other biological fluids). In the case of blood cell count (red blood cells, white blood cells and platelets), a peripheral blood smear can be made from a drop of blood and then observed under the microscope, but this sometimes yields inaccurate counts since it is very difficult to control the thickness of a blood smear. In order to get a very accurate concentration, it is necessary to know very precisely the volume of fluid being observed. Getting this kind of precision has long been very difficult to achieve (and hence expensive), therefore haemocytometers are conventionally non-disposable devices. They are typically made of a thick ‘H’-shape glass part, machined and polished with high precision (expensive process). Typically, an end user positions a cover slip onto the main glass part (resting on the protrusions of the H-shaped structure) to close the channel.

In use, a sample is introduced in between the glass part and the cover slip. Typically, the thinnest channels used were about 50 to 200 micrometers. At this channel depth, using a raw blood sample, there are too many cells stacked on top of each other to be able to image them individually. A typical white blood cell has a diameter in the neighborhood of 7 to 30 micrometers. Hence the blood must be diluted for useful imaging, and the diluting must be done by trained laboratory personnel. Further, this process typically does not employ in-situ staining of the cells, staining may be performed via pre-processing, e.g., during dilution with a staining reagent.

Additionally, the microscope depth of field (depth within which the objects observed are in focus) is typically only a few microns, which is much thinner than the depth of the channel. As a consequence, the imaging system must scan not only in the x and y directions (the dimensions of the image), but also in z direction (in the direction of the channel thickness).

Today, many laboratories perform blood counts using flow-cytometers that utilize laser diffraction by the cells in a jet of fluid (e.g., medical diagnostic devices made by Sysmex Corporation). These machines are large and expensive, and therefore not compatible with at-home use.

Analysis Device Capabilities

Analysis devices disclosed herein may provide improved sample capture and imaging. They can be manufactured with an extremely thin channel, of very tightly controlled dimensions, in which the cells are stained in-situ at low cost. Further end-users can use the devices and perform tests themselves. In certain embodiments, the devices use extremely thin adhesive tapes, such as those originally designed for the electronics industry. Such tapes have been used for, e.g., maintaining components in very thin smartphones. Using a very thin channel, as afforded by using such tape, the disclosed devices can be used without dilution and yet provide a suspension in which the cells are sufficiently spread out for clear imaging with little effort.

The disclosed analysis devices are appropriate for capturing and imaging suspensions of white blood cells, platelets, and/or other particle types or biological sample features. For purposes of illustration, the following discussion will focus on white blood cell applications, but unless otherwise indicated, the discussion applies to a variety of particle types. In certain embodiments, such analysis devices meet one or more of the following criteria.

1. Provide individually imageable and differentiable white blood cells in an imaging area.

2. Provide the white blood cells in the imaging area with sufficient density to provide an image having information content allowing a human or an algorithm to effectively count and classify them. For example, white blood cell concentrations from 0.1 k Cells/microliter (i.e., 100 Cells/microliter) to 100 k Cells/microliter may yield white blood cell surface densities from about 1 to 1000 cells/square millimeter or about 10 to 1000 cells/square millimeter.

3. In various embodiments, the white blood cells are provided in a monolayer (or approximately a monolayer). Typically, most or substantially all the white blood cells in a monolayer are not hidden or covered by other blood content and their morphological features appear clearly and are only limited by optical resolution.

4. The device can be used with different types of blood: whole blood from a direct prick, whole blood drawn in a tube, and/or preprocessed whole blood (e.g., post-centrifuge fractions or pre-stained). In the case of direct prick blood, and from patient's perspective, the device provides convenient sample drawing by capillary effect. In various embodiments, the patient is not required to provide more than a small amount of blood, e.g., about 1-15 microliters. Additionally, the patient should not have to hold their finger or other part of their body from which the blood is being drawn against the device for longer than a defined, relatively short period of time, e.g., less than about 15 seconds (or less than about 2 seconds).

5. Stain, fix, and/or lyse the cells as appropriate to facilitate imaging and image analysis.

Analysis Device Structure

Capillary Channel

The device includes a capture and image region in which the suspension forms. This region is sometimes referred to herein as a “capillary channel.” In certain embodiments, this region is relatively flat and thin, having a height, which is the smallest dimension, sized to hold approximately a monolayer of the sample features to be imaged, e.g., white blood cells. For embodiments employing white blood cells, this thickness may be between about 5 and 30 micrometers, or between about 10 and 15. Typically, the height of the capillary channel is measured in the same direction or substantially same direction as the direction of imaging. For example, if the imaging apparatus illuminates the capillary channel, the height of the capillary channel may be measured in a direction that is substantially parallel to the direction of illumination for the imaging apparatus.

Viewed from the direction of imaging, the capillary channel may have any of various shapes such as curved (e.g., circular or elliptical), polygonal (e.g., triangular or rectangular), or combinations thereof. The width and length of the capillary channel, also viewed from the direction of imaging, should be sufficiently large to allow a relatively large number of the sample features to occupy it in the monolayer. For example, if the shape is rectangular, the capillary channel may have a width and a length, each of between about 1 and 40 millimeters, or between about 5 and 15 millimeters. The length and width need not have the same dimensions. If the shape is rounded, e.g., circular, the capillary channel may have a diameter of between about 2 and 40 millimeters, or between about 10 and 20 millimeters. In certain embodiments, the width and length are each equal to or longer than the width and length of a typical image field of view (e.g., about 0.5 to 3 mm by 0.5 to 3 mm). In some cases, an image area is about 1 mm by 1.5 mm. In one embodiment, the width and length are each greater than about 2 mm. In some cases, the analysis device is designed or configured to allow images to be captured at or near the center of the capillary channel.

The capillary channel may have an open end in order to let the air out as the fluid sample enters the channel. This opening is sometimes referred as an air vent. It may have any of various shapes and locations in the capillary channel, but it is generally downstream of an inlet to the capillary channel.

In certain embodiments, the features and dimensions of the disclosed device are suitable for forming a suspension of white blood cells. However, the basic design can be applied to providing suspensions for imaging features of other biological sample components such as platelets, pathogens, etc.

Capillary Channel Receptacle

The capillary channel is formed by a structure referred to here as a capillary channel receptacle. In many implementations, the capillary channel receptacle is sized and shaped to enclose and define the capillary channel. In certain embodiments, the receptacle has a shape similar to that of the capillary channel itself. In certain embodiments, one or more of the receptacle components is substantially larger than the capillary channel.

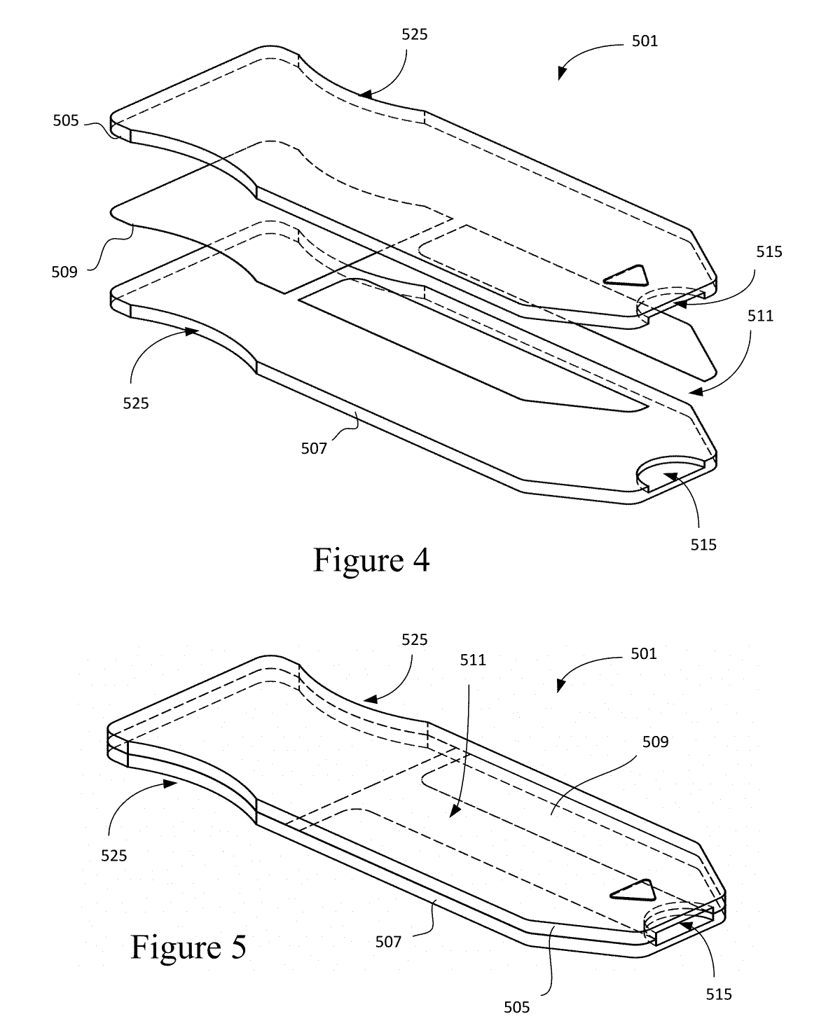

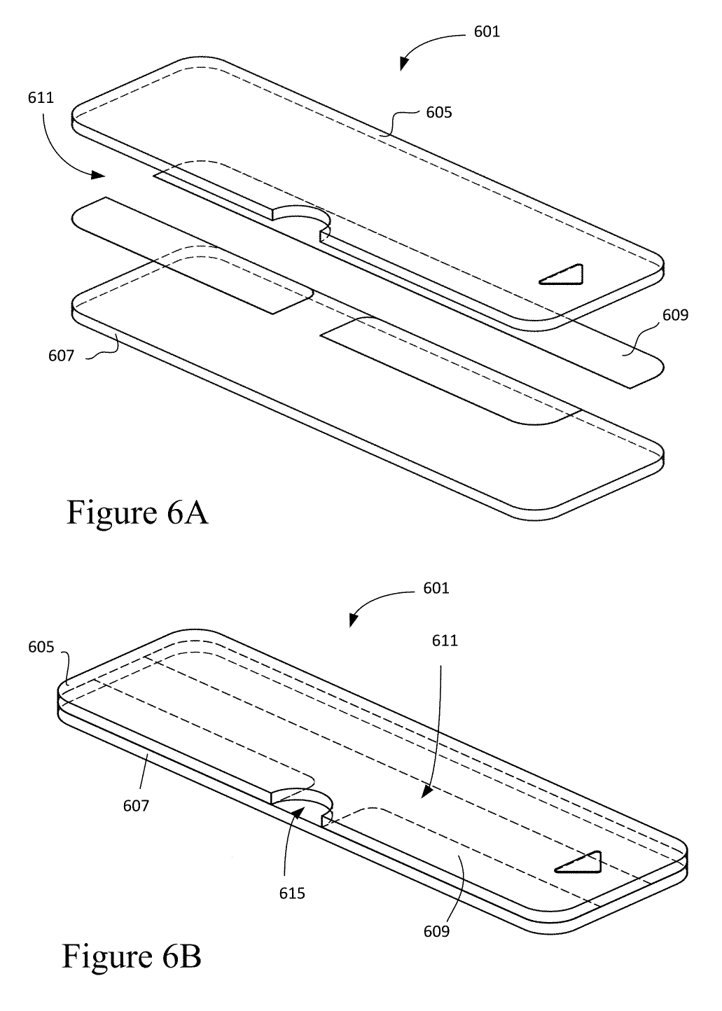

In certain embodiments, the receptacle is formed from a pair of substantially structures having substantially parallel facing surfaces. In various cases, the receptacle is formed from a pair of parallel plates, such as glass cover slips or glass microscope slides or other hydrophilic material, separated from one another by a separation structure having thickness that is the same as or substantially the same as the height of the capillary channel. In certain embodiments, the separation structure at least partially defines walls or edges of the capillary channel. For example, the separation structure may include walls parallel to the direction at which the biological sample is introduced into the capillary channel. See e.g., FIGS. 4 and 6A. In certain embodiments, the separation structure has a missing portion or center region that defines an area corresponding to some or all the capillary channel (e.g., the length and width, diameter, etc. of the capillary channel). Thus, the separation structure includes an inner perimeter, which defines the area of the capillary channel when viewed in the direction of imaging). The separation structure also includes an outer perimeter, which does directly define dimensions of the capillary channel. The distance between the inner and outer perimeters or edges may be constant or may vary azimuthally. In certain embodiments, the distance between the inner and outer perimeters or edges is, on average, between about 1 and 15 millimeters. Additionally, in certain embodiments, the separation structure has an opening between its center portion and the area outside. In some cases, this opening permits blood or other biological sample to move from outside the analysis device into the capillary channel. In certain embodiments, this opening has a width (direction perpendicular to the sample flow) of between about 1 and 10 millimeters. The separation structure may be provided in one or more pieces.

In certain embodiments, the separation structure has an opening other than the sample inlet. In some cases, it has one or more openings on a downstream side (away from the sample inlet side, in the direction of sample movement). In some cases, the separation structure is provided in two, three, or more pieces that collectively define a single capillary channel. For example, the separation structure may be defined by parallel pieces defining an upstream opening and a downstream opening. In some cases, a downstream opening provides a pressure release during blood flow; when the blood flows in it needs to push the air out. An example of such structure is shown in FIG. 8. The downstream opening is sometimes referred to as a vent. A vent need not be defined by shape and arrangement of the separation structure. In certain embodiments, a vent is provided by one or more openings in one or both of the parallel plates at a region in contact with the capillary channel.

The separation structure may be made from various materials such as a polymer (including elastomer), a glass (e.g., a silica glass), a metal, or a ceramic. In certain embodiments, the separation structure includes one or two adhesive surfaces, which may be used to hold the top and bottom plates in a single unit defining the capillary channel receptacle. In certain embodiments, the separation structure is or includes a double-sided adhesive tape. The separation structure has a thickness that allows the capillary channel to have a desired height. In certain embodiments, a double-sided adhesive separation structure has a nominal thickness of between about 10-30 micrometers, or between about 10-20 micrometers. The nominal thickness allows for slight variations in tape thickness. For example, in certain embodiments, such tape does not contain any thickness variations of more than about 500 nanometers over analysis devices from a given manufacturer's lot. As an example, a double-sided adhesive separation structure has a polyethylene terephthalate (PET) carrier. As a further example, a separation structure may have tackified acrylic adhesive layers on either side of the carrier.

The parallel plates of the capillary channel receptacle permit optical interrogation of a suspension formed in the capillary channel. Thus, one or both the plates are typically transparent or substantially transparent (e.g., having an optical transmissivity of at least about 50% in the region of the spectrum where imaging occurs). In certain embodiments, one or both of the plates is or includes a microscope cover slip or a microscope glass slide or similar structure that may be cut to a custom shape. In certain embodiments, the cover slips are a commercially available product such as VWR, available from Thermo Fisher Scientific, Inc. of Waltham, Mass. Suitable alternatives to glass coverslips include thin slips made from acrylic or other clear and hydrophilic plastic.

In some implementations, one or both of the parallel plates contains or is coated with a reagent that interacts with sample features to be imaged (e.g., white blood cells). In certain embodiments, the reagent is a stain for sample features. Examples of suitable stains for some applications include dyes such as methylene blue and/or cresyl violet. As an example of another reagent, a hemolysing agent such as saponin, may be used to lyse the red blood cells contacting the analysis device. Another coating may also be used to prevent certain types of cells from adhering to the surface of one or two of the plates. Such reagent(s) may be coated on the entire surfaces of the parallel plates or they may be coated on just certain regions. A glass spreader may be used to uniformly coat the slide. In other embodiments, the plates are dipped in a reagent that applies the coating(s).

Support Frame

In certain embodiments, the device includes a support frame that may serve any one or more of multiple functions associated with the construction and/or use of the device. In certain embodiments, the support frame has a thickness and/or a perimeter that is substantially larger than those of the capillary channel receptacle. This allows a medical care provider (e.g., a nurse or clinician) and/or a patient to easily handle the analysis device. It may also allow easy engagement with the imaging apparatus. In certain embodiments, the frame has a thickness, in the same direction (or approximately the same direction) as the height of the capillary channel, of between about 0.2 and about 2 millimeters, or between about 0.5 and 1.5 millimeters. In certain embodiments, including some illustrated below, the device does not include a support frame. Rather the capillary channel receptacle contains all the necessary structural components for an operable device. In such cases, the capillary channel receptacle may have structure with finger shaped and/or sized indentations or other structure allowing easy handling by the end user (clinician or patient). In certain embodiments, the frame-less structure provides structural stability that resists deformation and/or damage during user handling and during inflow of blood with the attendant pressure variations.

Because the support frame is supporting and framing the capillary channel structure, it should engage with the capillary channel structure without substantially interfering with imaging of sample features in the capillary channel. In this regard, the support frame may have a window, which may have one or more than one transparent layers. Typically, the window has a hydrophilic surface for contacting the sample. In certain embodiments, the frame is a hollow or donut-shaped structure, sized and shaped to surround or partially surround the capillary channel receptacle. The hollow or interior region provides a window for imaging. The support frame may have any of various overall shapes such as curved (e.g., circular or elliptical) or polygonal (e.g., triangular or rectangular). In various embodiments, the shape and size of the support frame facilitate easy handling of the analysis device.

In certain embodiments where the support frame is rectangular, the frame has a width and a length, each of between about 10 and 50 millimeters, or between about 20 and 40 millimeters. If the support frame shape is circular, the frame may have a diameter of between about 10 and about 50 millimeters, or between about 20 and 40 millimeters.

The frame may be constructed from one or more pieces. If constructed of two or more pieces, the pieces may be held together by any of various mechanisms such as clamping, adhesion, bonding, and the like. The frame and capillary channel receptacle may be glued together or otherwise connected in such a way that the blood or other sample cannot go in between the frame and the capillary channel; i.e., it is forced to go directly in the channel. In such embodiments, the two parts are tightly flushed against each other. In some embodiments, the connection is made by a very thin double-sided adhesive, or any other kind of adhesive. Also, the frame and the top part of the capillary channel receptacle may also be merged into one single piece of, e.g., glass, ceramic, or plastic. In certain embodiments, the combined frame and capillary channel receptacle part is made from a clear material, thus obviating the need for a separate window.

In certain embodiments, the frame is made from a material such as a polymer, ceramic, metal, and/or a glass. Examples of suitable polymers include acrylic (e.g., polymethyl methacrylate), polycarbonate, and the like. In various embodiments, the frame is non-reflective and opaque. In some embodiments, the frame is transparent.

Reservoir for Receiving a Sample

In certain embodiments, the analysis device includes a notch or indentation (sometimes referred to as a reservoir) on or near its outer perimeter, which reservoir facilitates contact of the device with a portion of the patient's body from which blood is drawn. The reservoir may be a feature of one or more components of the frame and/or the capillary channel receptacle. In certain frame-less embodiments (described elsewhere herein), the reservoir is present solely in the capillary channel receptacle. In practice, a nurse, clinician, or the end-user places the reservoir against the patient's finger or other appendage so that the device could draw blood from a wound by capillary action. In certain embodiments, the reservoir can host a drop of blood of about 5 to 10 microliters (e.g., about 8 microliters). In certain embodiments, the reservoir has a triangular shape, with the apex pointing inward, toward the capillary channel receptacle and away from the outer perimeter of the device. In certain embodiments, the reservoir has a concave semicircular shape.

The reservoir may be sized and shaped to rapidly draw a drop of blood away from the patient (e.g., in less than about a second), at which point the patient can move their finger (or other body portion) away from the analysis device. After that, the blood can be further drawn into capillary channel from the reservoir, over a longer period (e.g., about 30 to 60 seconds). Hence the patient does not need to hold their finger against the analysis device for the time required to form the thin suspension.

As indicated, the reservoir is provided in a manner that facilitates the initial capture and holding of the blood as it begins being draw into the capillary chamber by capillary force. In certain embodiments, the reservoir is constructed from a notch in one or both of a top and bottom parallel plates of the capillary channel receptable and/or in the frame, if present. Such notch(es) may be located anywhere along the perimeter of the device such as along the front or side of the analysis device. In some cases, the notch is located at the center or edge/corner of the front side of the analysis device. Some patients have a preference for placing analysis device corners against their wounds. In general, the notch may be located anywhere along any side of the analysis device as long as the reservoir is directly connected to the inlet of the capillary channel. Also, it should be understood that the inlet may not be at the center of the capillary channel if the channel has a T-shape for instance.

The notches forming the boundaries of a reservoir may be located on one more device components. For example, a notch can be located on either on one of the parallel plates that define the capillary channel receptacle, or on both of the plates, or on the frame (if there is one), or on one or both of the plates and the frame. If the reservoir is formed from both plates (top and bottom), then, in certain embodiments, at least one of the plates is provided as a support for temporarily holding the sample (as opposed to a through hole). This facilitates holding the blood (as in a reservoir) next to the capillary channel, rather than allowing it to escape from the device.

Examples of Analysis Devices

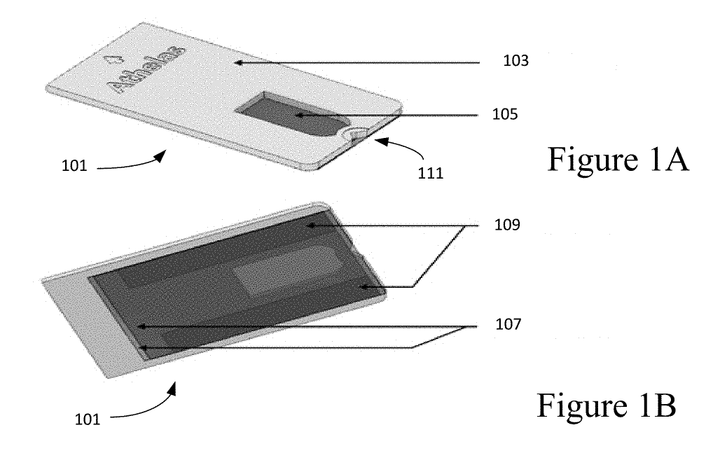

FIGS. 1A-1B, 2, and 3 depict examples of analysis devices containing a frame. FIGS. 1A and 1B show perspective views of a fully assembled framed analysis device 101 taken from above the example analysis device (FIG. 1A) and a perspective view taken from the underside of the analysis device (FIG. 1B). As can be seen, the analysis device includes a support frame (e.g., a white acrylic frame) 103 with an imaging window 105 over a capillary channel receptacle constructed of two dye stained parallel plates 107 (e.g., glass coverslips) that are adhered to one another using two parallel strips of double-sided tape 109 (an example of a separation structure). The window 105 is sized to allow sufficient imaging area (constrained by the objective lens magnification and the statistical distribution of the cells in the biological specimen). Typically, the window is between about 5 and 20 millimeters long. The capillary channel has two walls defined by strips of the adhesive tape 109, while the top and bottom walls are defined by glass coverslips 107.

The notch 111 shown on the right side of the devices shown in FIG. 1A provides a reservoir where the blood or other biological sample is received and temporarily held while it is being drawn in by capillary action. In certain embodiments, the reservoir is sized such that it can host a drop of blood, e.g., about 3 to 10 microliters.

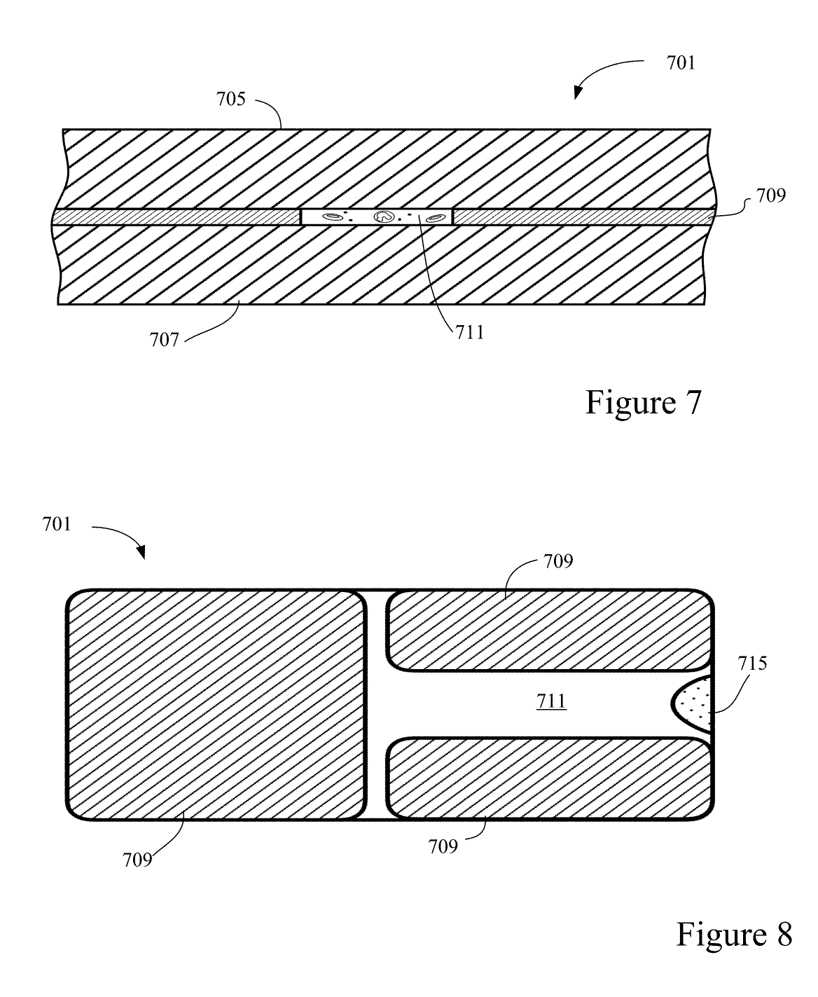

FIG. 7 shows a cross-sectional side view in which a device is cut through adhesive tape (separation structure 709). Two parallel plates 705 and 707 (e.g., coverslips) are separated by the capillary channel 711.

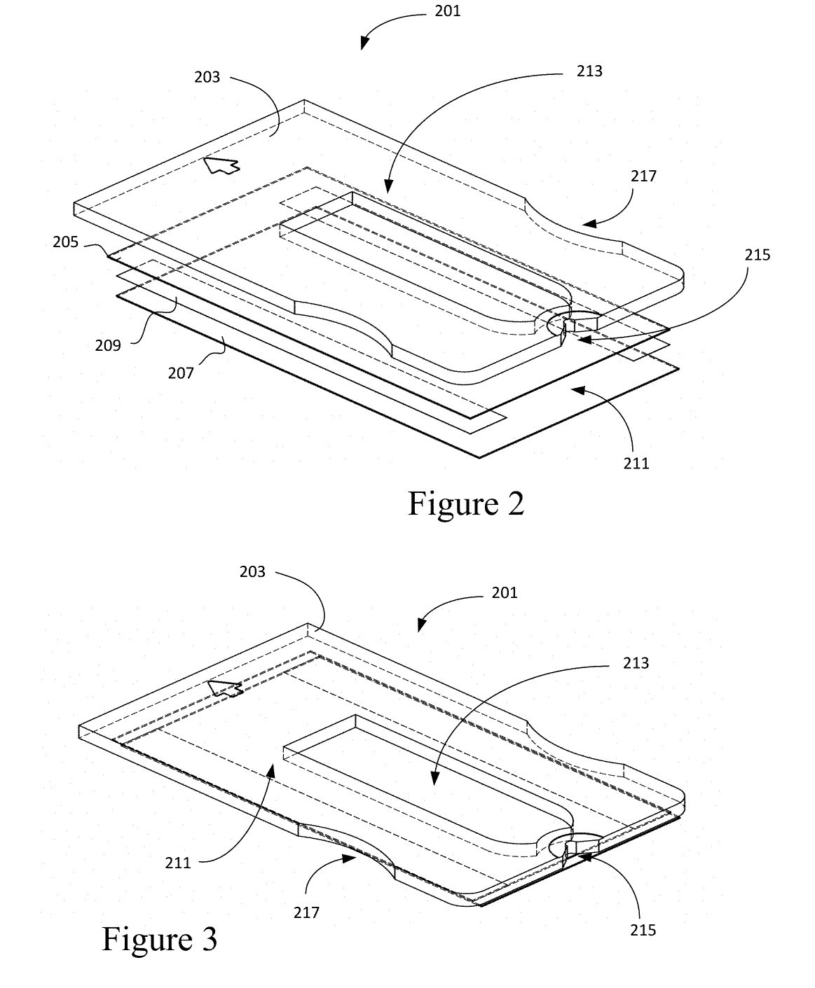

FIGS. 2 and 3 provide exploded and assembled views, respectively, of transparent analysis device components, in accordance with certain embodiments. As shown, an analysis device 201 includes a frame 203, a first parallel plate 205, a separation structure 209, and a second parallel plate 207, sandwiched in that order. The parallel plates 205 and 207 together with the separation structure 209 provide components of a capillary channel receptacle that defines boundaries of a capillary channel 211. The separation structure may be a doubled sided adhesive or a thin layer of other material having a thickness that allows the capillary channel to have a thickness as described herein. In general, the frame, separation structure, and parallel plates may have properties as described elsewhere herein.

In the depicted embodiment, a window is provided by an opening 213 in frame 203. In certain embodiments, at least the first parallel plate 205 is substantially transparent to allow imaging of a suspension via the window. The suspension is formed in the capillary channel within an opening in the separation structure 209 and between the parallel plates 207 and 209. The sample is provided to the analysis device 201 via a reservoir 215. Indentations 217 in frame 203 facilitate grasping and holding the analysis device.

A reservoir may be provided on an edge of an analysis device such as is depicted by reference numeral 215 in FIGS. 2 and 3. In certain embodiments, a reservoir is defined by a notch in a frame of an analysis device. In some cases, the notch has a semicircular or a triangular shape. This is not essential. The notch can have any shape, curved, straight, combined, etc. In various embodiments, the notch is sized and shaped to facilitate contact with a finger or other body portion that provides blood or other biological sample.

Notches defining reservoirs may be defined in a top plate, a bottom plate, or both, in which case the notches are co-extensive or overlap with one another. This structure in which one plate of a capillary channel receptacle has a notch and the other does not (or at least not one as big as the first one) allows the biological sample to sit as a pool at the edge of the capillary channel while it is drawn in by capillary force.

In certain embodiments, the top plate and/or the bottom plate has a thickness that is substantially greater than the height of the capillary channel (e.g., about 1 mm vs. 10 um). The thin capillary channel allows the analysis device to quickly suck in the sample. In certain embodiments, one or both plates have a thickness of between about 0.1 and 1.5 millimeters (or between about 0.5 and 1 millimeter). When the plate material is sufficient rigid (e.g., glass), it substantially resists bending due to a pressure drop when the blood or other sample flows in.

In certain embodiments, the separation structure is not divided into two simple strips but is provided in a more complex shape. This may constrain the amount of blood or other sample that flows in to a given volume. In some embodiments, the separation structure includes an orthogonal piece that prevents the blood or other sample from flowing further. When the sample flows in, it can push the air out via vents defined in the separation structure and/or through one or both of the top and bottom plates. In certain embodiments, the separation structure has a thickness of between about 5 and 30 micrometers, or between about 10 and 15 micrometers.

FIGS. 4 and 5 illustrate exploded and assembled views, respectively, of transparent analysis device components without a frame in accordance with certain embodiments. In some ways, this embodiment is similar to the embodiment of FIGS. 2 and 3, but it does not include a frame.

As shown, an analysis device 501 includes a first parallel plate 505, a separation structure 509, and a second parallel plate 507, sandwiched in that order. The parallel plates 505 and 507 together with the separation structure 509 provide components of a capillary channel receptacle that defines boundaries of a capillary channel 511. Further, any one or more of these components provides sufficient structural rigidity and strength to allow handling without a frame.

The device of the disclosed embodiment has a reservoir 515 on a front side of the analysis device. Further, the device has indentations 525 to facilitate gripping and handling of the device. The overall shape of the analysis devices bears a resemblance to an arrow head. By comparison, the device of FIGS. 6A and 6B has a reservoir on the edge of the analysis device and no indentations. Various combinations of reservoir location and indentation are possible.

FIGS. 6A and 6B illustrate exploded and assembled views, respectively, of transparent analysis device components without a frame in accordance with certain embodiments. As shown, an analysis device 601 includes a first parallel plate 605, a separation structure 609, and a second parallel plate 607, sandwiched in that order. Similar to embodiments depicted in FIGS. 2 and 5, the parallel plates 605 and 607 together with the separation structure 609 provide components of a capillary channel receptacle that defines boundaries of a capillary channel 611. Further, any one or more of these components provides sufficient structural rigidity and strength to allow handling without a frame. As mentioned, the disclosed embodiments have a reservoir 615 on a side of the analysis device.

As with other embodiments, the separation structure in the embodiments of FIGS. 4-5 and 6A-6B may be a doubled sided adhesive or a thin layer of other material having a thickness that allows the capillary channel to have a height as described herein. Further as with the other depicted embodiments, the separation structure, and parallel plates may have properties as described elsewhere herein.

Also in the embodiments of FIGS. 4-5 and 6A-6B, at least the first parallel plate is substantially transparent or otherwise serves as a window. During operation, a thin suspension is formed in the capillary channel within an opening 515 or 615 in the separation structure and between the parallel plates.

FIGS. 7 and 8 present a generalized representation of an analysis device 701 in accordance with certain embodiments. FIG. 7 presents a cross-sectional view and FIG. 8 presents a top view of the device. As shown, an analysis device 701 includes first and second parallel structures 705 and 707, and interposed between them a separation structure 709. The parallel structures 705 and 707 together with the separation structure 709 define boundaries of a capillary channel 711. As with various other embodiments, the separation structure 709 may be a doubled sided adhesive or a thin layer of other material having a thickness that allows the capillary channel to have a uniform height. Further as with the other depicted embodiments, the separation structure, and parallel plates may have properties as described elsewhere herein. The first parallel plate may be substantially transparent or otherwise serves as a window to permit imaging. During operation, an imageable portion of a sample is formed in the capillary channel 711. In FIG. 7, cells or other biological features (e.g., red blood cells, white blood cells, platelets, etc.) are illustrated in channel 711. The depicted device also has a reservoir 715 for receiving blood or other samples and allowing it them to be drawn into the capillary channel. The reservoir may be created by various techniques such as by forming a notch in one or both of the first and second structures.

Methods of Fabricating the Device

In certain embodiments, an analysis device is fabricated as follows. The components of the support frame, if used, and capillary channel receptacle are obtained or fabricated (e.g., molded, cut or machined to the necessary dimensions). The starting components for the capillary channel receptacle may include transparent coverslips or other parallel plates, and double-sided adhesive tape or other material for the separation structure. The tape may be cut to appropriate size or shape to define the sidewalls of the capillary channel. The coverslips or other hydrophilic sheets (e.g., glass slides) may be machined or obtained commercially (as cover glass, glass coverslips, or plastic coverslips). The glass plates are adhered to one another by appropriately positioned double-sided tape. As a result the capillary channel receptacle is formed.

The application of the dye (stain) or other reagent(s) can be applied to device components in a various ways. Examples include spraying (including airbrushing), jet printing, dunk staining (in a bath containing the stain), and the like. The reagent(s) may be applied to selected surfaces (e.g., one or both facing parallel surfaces of the plates) or to the entire components (e.g., by dunking both plates in a bath of reagent).

In one example, a small quantity of stain (e.g., about 5 uL of the dye in liquid form) is delivered in front of the capillary channel in a very viscous form. In another example, a small quantity (e.g., about 2 uL) of the stain or other reagent is taken up by the capillary channel by putting one end of the capillary channel into the stain. In another example, the stain or other reagent is smeared across the coverslip by a traditional smearing mechanism (e.g., placing a small quantity of the reagent on the sheet, forming a wedge of the reagent between the sheet and a second slide, and dragging the second slide over a face sheet to evenly smear the reagent over the face of the sheet). These examples assume that the dye is provided in the form of a liquid. For example, the dye may be smeared or coated as a methanol-based solution, and the liquid content allowed to evaporate and leave behind solid content on the surface in the imaging region. However, in some embodiments, a dye is provided as a powder that may be applied as a dry coating. Regardless of whether the dye is provided in a wet or dry form, it forms a coating. In some embodiments, the dye is applied as a powder or as an oil based solution in the inlet of the channel only, and the blood will mix with it.

In certain embodiments, the support frame is fabricated by laser cutting an acrylic sheet and then applying markings by laser etching or silk screening. In certain embodiments, the device is constructed by molding, e.g., injection molding. When molded, the height of the channel may be defined by the mold of the receptacle rather than by a separation structure such as a separate layer of tape. In certain embodiments, the capillary channel receptacle is molded as a single part. In certain embodiments, the capillary channel receptacle is separately molded as two sub-components. In this case, the two sub-components need to be bonded together by a suitable bonding technique such as: gluing, adhesion, and ultra-sound, melting. In either case, the shape, dimensions, and arrangement of features may be as described above for any of the figures presented herein.

Methods of Using the Device

As an example of how to use the device, a blood test is described. In related examples, other biological samples are tested. Initially, an incision is made in the patient, e.g., the patient is lanced. The patient then places the lanced portion of their body (e.g., a fingertip) against the notch/reservoir of the analysis device for a first duration. Then the patient then moves her body out of contact with the analysis device. During this short first duration some of the blood enters the analysis device but does not penetrate deeply into the capillary channel. The blood is temporarily held in the reservoir. The blood then penetrates more deeply into the capillary channel (drawn by capillary action) to form a thin suspension during a second duration. The second duration is longer than the first duration. After the second duration, the suspension is suitable imaging and imaging can take place. In certain embodiments, the first duration is less than about 5 seconds, or less than about 2 seconds. In certain embodiments, the second duration is between about 30 and 180 seconds.

While the sample blood flows into the capillary channel (and contacts the coverslip), it interfaces with a stain and/or other reagent that has been dried on the surface of one or both coverslips. After the blood interfaces with the stain it is drawn into a capillary channel by capillary action, it forms an image-ready suspension, which may be a monolayer.

The analysis devices may be provided in a kit with other components such as lancets and cleaning brush for inside the imager's analysis device insertion slot.

### Terminology

The term “biological sample” refers to a sample, typically derived from a biological fluid, tissue, organ, etc., often taken from an organism suspected of having a condition such as an infection, neoplasm, mutation, or aneuploidy. Such samples include, but are not limited to sputum/oral fluid, amniotic fluid, blood, a blood fraction, fine needle biopsy samples (e.g., surgical biopsy, fine needle biopsy, etc.), urine, semen, stool, vaginal fluid, peritoneal fluid, pleural fluid, tissue explant, organ culture, cell culture, and any other tissue or cell preparation, or fraction or derivative thereof or isolated therefrom. The biological sample may be taken from a multicellular organism or it may be of one or more single cellular organisms. In some cases, the biological sample is taken from a multicellular organism and includes both cells comprising the genome of the organism and cells from another organism such as a parasite. The sample may be used directly as obtained from the biological source or following a pretreatment to modify the character of the sample. For example, such pretreatment may include preparing plasma from blood, diluting viscous fluids, culturing cells or tissue, and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. Such “treated” or “processed” samples are still considered to be biological samples with respect to the methods described herein.

Biological samples can be obtained from any subject or biological source. Although the sample is often taken from a human subject (e.g., a patient), samples can be taken from any organism, including, but not limited to mammals (e.g., dogs, cats, horses, goats, sheep, cattle, pigs, etc.), non-mammal higher organisms (e.g., reptiles, amphibians), vertebrates and invertebrates, and may also be or include any single-celled organism such as a eukaryotic organism (including plants and algae) or a prokaryotic organism, archaeon, microorganisms (e.g. bacteria, archaea, fungi, protists, viruses), and aquatic plankton.

In various embodiments described herein, a biological sample is taken from an individual or “host.” Such samples may include any of the cells of the host (i.e., cells having the genome of the individual) or host tissue along with, in some cases, any non-host cells, non-host multicellular organisms, etc. described below. In various embodiments, the biological sample is provided in a format that facilitates imaging and automated image analysis. As an example, the biological sample may be stained and/or converted to a thin suspension before image analysis.

Host—An organism providing the biological sample. Examples include higher animals including mammals, including humans, reptiles, amphibians, and other sources of biological samples as presented above.

Sample Feature—A sample feature is a feature of the biological sample that represents a potentially clinically interesting condition. In certain embodiments, a sample feature is a feature that appears in an image of a biological sample and can be segmented and classified by a machine learning model. Examples of sample features include cells of a host (e.g., white blood cells, platelets, and red blood cells) and parasitical organisms (e.g., protozoa, bacteria, and viruses).

Suspension—a thin layer of blood or other biological fluid sample provided in a form that facilitates imaging to highlight sample features that can be analyzed to automatically classify the sample features. Often a suspension is provided on a substrate that facilitates conversion of a raw biological sample taken from a host to a thin image-ready form (the suspension). In certain embodiments, the suspension has a thickness of at most about 50 micrometers or at most about 20 micrometers. In some embodiments, suspension thickness is between about 5 and 30 micrometers. In various embodiments, the suspension presents cells, multicellular organisms, and/or other features of biological significance in a monolayer, such that only a single feature exists (or appears in an image) at any x-y position in the image. It is often desirable that the suspension present sample features in a form that can be imaged with sufficient detail that an image analysis routine can reliably classify the sample features. In many embodiments, the suspension presents sample features with sufficient clarity to resolve the entire boundaries of the sample features and some interior variations within the boundaries.

Suspensions of biological samples may be produced in any of various ways using various types of apparatus, some known to those of skill in the art and others novel. Examples include the analysis devices illustrated in the figures. Generally, such analysis devices produce a suspension by simply touching a drop of blood or other sample to one side of the analysis device and allowing capillary action to draw the sample into a region where it distributes in a thin layer constituting the suspension. In certain embodiments, an analysis device serves as both a reaction chamber for preprocessing the sample prior to imaging and a container for imaging a suspension of the sample. Examples of reactions that can take place on the analysis device include staining one or more components of the sample (e.g., host cells and/or parasites) and lysing one or more cells of the sample. In some embodiments, lysing is performed on cells that could interfere with the image analysis.

### OTHER EMBODIMENTS

The foregoing description of the specific embodiments explains the general nature of the embodiments herein such that others can, by applying current knowledge, readily modify and/or adapt for various applications such specific embodiments without departing from the generic concept, and, therefore, such adaptations and modifications are within the scope of the disclosed embodiments. It is to be understood that the phraseology or terminology employed herein is for the purpose of description and not of limitation. Therefore, those skilled in the art will recognize that the embodiments herein can be practiced with modification within the spirit and scope of the claims as described herein. For example, while most examples described operate on blood or other liquid biological samples, the disclosure is not so limited. In certain applications, the disclosed embodiments are employed in various types of medical diagnostics, air-quality analysis, biopsies, etc.

It is Applicant's intent that, unless a claim element uses “means” or “step,” it should not invoke 35 U.S.C. § 112(f).

What is claimed is:

1. An analysis device for producing an imageable suspension of a liquid biological sample, the analysis device comprising:
a capillary channel having (i) an inlet for receiving the biological sample, (ii) a height sized to form a thin suspension of the biological sample when the biological sample enters the capillary channel, wherein the height is between about 5 and 30 micrometers, and (iii) a width, in a direction substantially perpendicular to flow of the biological sample at the inlet, of between about 1 and 40 millimeters;
a substantially transparent window defining a surface of the capillary channel and configured to form a suspension suitable for imaging the biological sample through the substantially transparent window; and
a dye coated on at least a portion of the substantially transparent window and/or another surface of the capillary channel, wherein the dye stains a particular cell type from the biological sample when the biological sample contacts the dye.

2. The analysis device of claim 1, wherein the height of the capillary channel is between about 10 and 15 micrometers.

3. The analysis device of claim 1, wherein the height of the capillary channel is sized to form a monolayer of cells in the biological sample.

4. The analysis device of claim 1, wherein the capillary channel further comprises a length, in a direction substantially parallel to a direction of flow of the biological sample at the inlet, of between about 5 and 40 millimeters.

5. The analysis device of claim 1, wherein the substantially transparent window comprises more than one transparent layers.

6. The analysis device of claim 1, wherein the substantially transparent window has a thickness of between about 0.1 and 1.5 millimeters.

7. The analysis device of claim 1, further comprising a substantially flat surface, wherein the substantially transparent window and the substantially flat surface are disposed parallel to one another and separated by the height of the capillary channel to thereby partially enclose the capillary channel.

8. The analysis device of claim 7, further comprising a separation structure for separating the substantially transparent window and the substantially flat surface by the height of the capillary channel.

9. The analysis device of claim 8, wherein the separation structure comprises double-sided adhesive tape.

10. The analysis device of claim 1, further comprising a support frame for supporting the substantially transparent window and the capillary channel without interfering with imaging the suspension.

11. The analysis device of claim 1, wherein the analysis device does not have a support frame.

12. The analysis device of claim 11, wherein the substantially transparent window has one or more indentations sized and shaped to permit grasping with the fingers.

13. The analysis device of claim 1, wherein the dye comprises methylene blue and/or cresyl violet.

14. The analysis device of claim 1, further comprising a lysing agent for one or more cells present in the biological sample, wherein the lysing agent is provided on at least a portion of the substantially transparent window and/or the other surface of the capillary channel.

15. The analysis device of claim 14, wherein the lysing agent comprises a hemolysing agent.

16. The analysis device of claim 1, further comprising a reagent that prevents certain types of cells from adhering to at least a portion of the substantially transparent window and/or the other surface of the capillary channel.

17. The analysis device of claim 1, further comprising a reservoir configured to receive the liquid biological sample, wherein the reservoir comprises a notch in (i) one or two parallel plates defining a portion capillary channel, and/or (ii) a frame of the analysis device, and wherein the reservoir is fluidically coupled to the inlet for receiving the biological sample.

18. A method of producing and/or using a suspension comprising applying a biological sample to an analysis device comprising:
a capillary channel having (i) an inlet for receiving the biological sample, (ii) a height sized to form a thin suspension of the biological sample when the biological sample enters the capillary channel, wherein the height is between about 5 and 30 micrometers, and (iii) a width or diameter, in a direction substantially perpendicular to flow of the biological sample at the inlet, of between about 1 and 40 millimeters;
a substantially transparent window defining a surface of the capillary channel and configured to form a suspension suitable for imaging the biological sample through the substantially transparent window; and
a dye coated on at least a portion of the substantially transparent window and/or another surface of the capillary channel, wherein the dye stains a particular cell type from the biological sample when the biological sample contacts the dye.

19. The method of claim 18, further comprising imaging the suspension to produce an image of the suspension.

20. The method of claim 19, further comprising applying the image of the suspension to an image processing algorithm to classify and/or count the cellular artifacts from the suspension.

21. The method of claim 19, wherein applying a biological sample to an analysis device comprises causing a patient to contact a wound with a reservoir on the analysis device.
